(19) United States
(12) Patent Application Publication
(10) Pub. No.: US 2023/0035823 A1
(43) Pub. Date: Feb. 2, 2023

(54) ANTIHISTAMINE AND ANTI-MICROBIAL NASAL SOLUTIONS AND RELATED METHODS

(71) Applicant: Xlear, Inc., Orem, UT (US)

(72) Inventors: Nathan Jones, Orem, UT (US); Alonzo H. Jones, Plainview, TX (US)

(21) Appl. No.: 17/877,937

(22) Filed: Jul. 30, 2022

Related U.S. Application Data
(60) Provisional application No. 63/227,867, filed on Jul. 30, 2021.

(51) Int. Cl. A61K 9/00; A61K 31/496; A61K 31/445; A61K 31/7004; A61K 31/135; A61P 31/04

(52) U.S. Cl. CPC A61K 9/0073; A61K 31/135; A61K 31/445; A61K 31/496; A61K 31/7004; A61P 31/04

(57) ABSTRACT

Compositions and methods for alleviating an allergy condition while also reducing a drying effect of an antihistamine and/or treating an infection or at least reducing the prevalence of bacteria or another infectious microorganism. In some embodiments and implementations, a composition may be provided comprising an antihistamine comprising at least one of brompheniramine, cetirizine, clemastine, diphenhydramine, fexofenadine, and loratadine, preferably in a therapeutically effective amount for treating the allergy condition in the human having the allergy condition. The composition may further comprise an anti-microbial and/or anti-drying agent comprising one or more non-hexose, sugar alcohols, such as at least one of xylitol, xylose, erythritol, erythrose, ribose, ketose, and/or arabinose in one or more therapeutically effective amounts for reducing nasal dryness caused by the allergy treatment composition, treating an infection, and/or reducing the prevalence of a microbial organism in a subject’s nasal passage and/or sinus cavities.

### RELATED APPLICATIONS

This application claims the benefit under 35 U.S.C. § 119(e) of U.S. Provisional Pat. Application No. 63/227,867 filed Jul. 30, 2021, and titled “ANTIHISTAMINE AND ANTI-MICROBIAL NASAL SOLUTIONS AND RELATED METHODS,” which application is incorporated herein by reference in its entirety.

### SUMMARY

Disclosed herein are examples of embodiments and implementations of compositions and methods for eliminating or at least reducing unwanted microbial populations in nasal passages and/or sinus cavities, including but not limited to treatment of infections, such as nasal infections, sinus infections, ear infections and/or lung infections, while simultaneously treating or preventing an allergy condition using an antihistamine and/or reducing the negative side effects of the antihistamine, such as dryness. Compositions including antihistamines and antimicrobial agents, such as xylitol or other preferably non-prescription antibiotic agents are also disclosed.

In some embodiments, a nasal solution for treating, alleviating, or preventing an allergy condition while simultaneously treating an infection, preventing an infection, and/or eliminating or at least reducing an unwanted microbial population within a patient’s nasal passage and/or sinus cavity, may comprise an antihistamine in an amount effective for treating or preventing an allergy condition, along with at least one non-hexose, sugar alcohol, such as at least one of xylitol, xylose, erythritol, erythrose, ribose, ketose, and/or arabinose in an amount effective for treating or preventing an infection and/or effective for eliminating or at least counteracting the drying effect of the antihistamine. The antihistamine may comprise one or more of brompheniramine, cetirizine, clemastine, diphenhydramine, fexofenadine, and loratadine.

In some embodiments, the composition may comprise a nasal solution. In such embodiments, the nasal solution may comprise a nasal spray, and may further comprise a nasal spray bottle configured to deliver the nasal solution. Alternatively, the nasal solution may comprise a nasal dropper configured to deliver the nasal solution in a liquid drop form. In other embodiments, the nasal solution may comprise a gel.

In some implementations of methods for treating or preventing an allergy condition and simultaneously reducing the drying effect of the antihistamine and/or treating or preventing an infection by reducing or eliminating bacteria or another microbe or infectious microorganism, the method may comprise identifying a subject having an allergy condition and/or an infection and providing a solution. The solution may comprise a composition including an antihistamine in an amount effective for treating the allergy condition and at least one of xylitol and xylose in an amount effective for reducing the drying effect of the antihistamine and/or effective for reducing or eliminating the bacteria or other agent causing the infection. Such methods may further comprise delivering the solution into at least one of the subject’s nose, eyes, mouth, and throat.

In some implementations, the solution may comprise a nasal solution, and, in such implementations, the step of delivering the solution may comprise delivering the nasal solution into the subject’s nose.

In a more particular example of a method for treatment of a bacterial infection using a nasal composition according to some implementations, the method may comprise identifying a subject having a bacterial infection and delivering one or more doses of a composition into the subject’s nasal passage. The composition may comprise an antihistamine comprising at least one of brompheniramine, cetirizine, clemastine, diphenhydramine, fexofenadine, and loratadine and at least one of xylitol and xylose.

In some implementations, the antihistamine may be present in the composition in a therapeutically effective concentration for treating an allergy condition in the subject. The antihistamine may be present in the composition in a concentration of between about 0.01% and about 2% by weight. In some more preferred implementations, the antihistamine may be present in the composition in a concentration of between about 0.2% and about 2% by weight.

In some implementations, the at least one of xylitol and xylose may be present in the composition in a therapeutically effective concentration for treating the bacterial infection. In some such implementations, the at least one of xylitol and xylose may be present in the composition in a concentration of between about 5% and about 20% by weight.

In some implementations, the bacterial infection may comprise a bacterial biofilm. In some such implementations, the composition may be used to disrupt the bacterial biofilm. In some such implementations, the composition may be used to eliminate the bacterial biofilm altogether.

In some implementations, the dose may be part of a treatment regimen. For example, the treatment regimen may comprise delivering the composition in each nostril of the subject three times daily, in some cases for at least ten days.

In an example of a method for treatment of a bacterial infection using a nasal composition according to some implementations, the method may comprise identifying a subject having a bacterial infection, wherein the bacterial infection comprises a bacterial biofilm. One or more doses of a composition may be delivered into the subject’s nasal passage. The composition may comprise an antihistamine comprising at least one of brompheniramine, cetirizine, clemastine, diphenhydramine, fexofenadine, and loratadine; and at least one non-hexose sugar alcohol in a in a therapeutically effective concentration for treating the bacterial infection.

In some implementations, the at least one non-hexose sugar alcohol may comprise at least one of xylitol, xylose, erythritol, erythrose, ribose, ketose, and arabinose.

In some implementations, the at least one non-hexose sugar alcohol may be present in the composition in a concentration of between about 5% and about 20% by weight.

In some implementations, the at least one non-hexose sugar alcohol may comprise xylitol. In some such implementations, the xylitol may be present in the composition in a concentration of at least 2% by weight. In some more preferred implementations, the xylitol may be present in the composition in a concentration of between about 5% and about 20% by weight.

In an example of a method for disruption of a bacterial biofilm using a nasal composition, the method may comprise identifying a subject having a bacterial infection comprising a bacterial biofilm; and delivering a dose of a composition into the subject’s nasal passage. In some implementations, the composition may comprise xylitol in a in a concentration of at least 2% by weight.

In some implementations, the composition may further comprise an antihistamine. In some such implementations, the antihistamine may comprise at least one of brompheniramine, cetirizine, clemastine, diphenhydramine, fexofenadine, and loratadine.

In some implementations, the xylitol may be present in the composition in a concentration of between about 5% and about 20% by weight

In some implementations, the composition may further comprise at least one of xylose, erythritol, erythrose, ribose, ketose, and arabinose.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are nonlimiting and non-exhaustive. Reference is made to certain of such illustrative embodiments that are depicted in the figures, in which:

FIG. 1 illustrates intranasal delivery of an antihistamine and antimicrobial composition including at least one non-hexose, sugar alcohol consistent with embodiments of the present disclosure.

### DETAILED DESCRIPTION

Antihistamines are widely used to alleviate allergy symptoms such as itching and a runny nose. Antihistamines may prevent histamines produced by the body’s immune response from attaching to histamine receptors in the nose and/or throat, thereby reducing allergy systems. Blocking this histamine response, however, has certain undesirable side effects. For example, administration of antihistamines and/or other anti-mucosal compositions may cause an individual’s nasopharynx, nose, and/or throat to become uncomfortably dry and such dryness may lead to further conditions and/or complications.

Consistent with embodiments disclosed herein, certain sugar alcohols, such as xylitol, may be administered in conjunction with certain preferred antihistamines to provide a synergistic effect, which may include treating or preventing an allergy condition, eliminating or preventing bacterial or other microbial colonies within the nasal passages and/or sinuses, and alleviating at least some of the negative side effects of the antihistamine(s), such as unwanted drying of the linings of the nasal passages and/or throat.

As an additional benefit, the synergistic compositions disclosed herein may avoid use of prescription antibiotics, which are known to cause infectious microorganisms to develop resistance over time and are often ineffective in treating certain kinds of bacterial infections, such as bacterial biofilms. Because chronic nasal infections, sinus infections, ear infections, lung infections and sinusitis are often caused by such biofilms, antibiotic treatment alone is of limited usefulness.

Thus, the compositions disclosed herein may be sold over the counter. In addition, the use of xylitol and/or other non-hexose sugar alcohols may reduce the ability of the bacteria to evolve resistance to the antibiotic agents. Also, avoiding prescription antibiotics may eliminate or at least reduce the potentially harmful impact of such antibiotics on the useful bacteria in the human biodome.

In addition, because typically free-floating bacteria (planktonic) do not develop resistance, the anti-adherence properties of xylitol and possibly other flexible, non-hexose sugar alcohols, can also provide improved ability to avoid antibiotic resistance. Antibiotic resistance is typically developed in response to quorum sensing. When bacteria are threatened, they release chemical signals and, when the signal gets strong enough, some of the bacteria in the biofilm increase their mutation rate, seeking a way to cope with the threat. Thus, the anti-adherence properties of xylitol, and possibly other related non-hexose, flexible sugar alcohols, may work against the development of antibiotic resistance by disruption of biofilms.

Furthermore, certain non-hexose sugar alcohols, such as xylitol, xylose, erythritol, ribose, and arabinose, for example, have a sweetness equivalent to that of sucrose, but also possess unique properties that render it unsuitable as a source of energy for certain bacteria and/or other microorganisms. More particularly, without being limited by theory, it is thought that, due to its five-carbon sugar alcohol structure, xylitol, for example, cannot be used as an energy source for most oral microorganisms but may be ingested or otherwise prevent ingestion of other similar energy sources. Regular consumption of xylitol has also been shown to reduce the incidence of dental caries. This is primarily attributed to xylitol’s ability to inhibit and/or reduce the growth and acid production of S. mutans, which is thought to be one of the more important bacterium taking part in the pathogenesis of dental caries.

Xylitol has also been demonstrated to inhibit the growth of Streptococcus pneumonia in vitro during its logarithmic growth phase. The Streptococcus pneumonia bacteria species is believed to be the causative agent of certain types of pneumonia and upper respiratory infections, and is also associated with other infectious diseases, such as meningitis and sepsis.

Xylitol has also been shown to be very effective in moisturizing nasal passages and the like. Without being limited by theory, this is thought to occur because xylitol can create a hyper-osmotic solution that pulls moisture towards it from surrounding tissues without generated mucous. Thus, the combination of xylitol, or other similar sugar alcohols disclosed herein, may result in a decrease in mucous production even when used with drying antihistamines without the accompanying dryness, or at least with reduced dryness, that typically accompanies antihistamines. By combining this with the accompanying anti-bacterial and other health benefits associated with xylitol and other similar agents, a synergistic composition is provided that may be useful in replacing a series of other pharmacological agents.

The present inventor has discovered that these unique characteristics make xylitol, and possibly other related sugar alcohols, extremely useful in disrupting bacterial biofilms. Again, without being limited by theory, it is thought that this may be due to the ability of these substances to be treated as a source of energy and taken in by bacteria (due to xylitol’s similar shape to other sugars that may serve as an energy source), which leaves no room for six-carbon sugars and thereby impedes bacterial growth and reproduction by, in essence, starvation in the presence of xylitol.

The present inventor has further discovered that, not only may xylitol, and possibly other related non-hexose, flexible sugar alcohols, be useful for starvation of bacteria, but they may also serve as a biofilm disrupter. Without being limited by theory, it is thought that this may happen by starvation of bacteria on the upper layer, after which the layer immediately below is starved, and so on until the biofilm has been suitably disrupted and/or eliminated. In fact, by providing xylitol or, in some embodiments, other similar sugar alcohols, prescription antibiotics may be avoided altogether, while still providing a beneficial impact to the patient on biofilm disruption.

In some embodiments and implementations, nasal, ear, lung, and/or sinus infection treatment compositions disclosed herein may comprise methods, agents, compositions, etc. disclosed in U.S. Pat. Nos. 6,054,143 and 6,258,372, both titled “XYLITOL NOSE SPRAY” and U.S. Pat. No. 6,599,883 titled “NASAL DELIVERY OF XYLITOL,” each of which is incorporated herein by reference in its entirety.

In some embodiments and implementations, administering antihistamines in conjunction with xylitol may reduce dryness associated with the use of the antihistamine compositions in an individual’s nasopharynx, nose, and/or throat that would normally otherwise be associated with use of the antihistamine composition alone. In some embodiments, polysaccharides, monosaccharides and/or sugar alcohol compositions utilized in the anti-mucosal compositions disclosed herein may be prepared, at least in part, utilizing methods disclosed in U.S. Pat. Nos. 6,054,143 and 6,258,372, both titled “XYLITOL NOSE SPRAY” and U.S. Pat. No. 6,599,883 titled “NASAL DELIVERY OF XYLITOL,” each of which was previously incorporated by reference in its entirety.

In preferred embodiments and implementations, the antihistamine may specifically be selected from one or more of brompheniramine, cetirizine, clemastine, diphenhydramine, fexofenadine, and loratadine. In some such embodiments and implementations, the antihistamine may be present in a concentration of between about 0.01% and about 2% by weight. More preferably, the antihistamine may be present in a concentration of between about 0.2% and about 2% by weight.

In some preferred embodiments and implementations, a threshold concentration of one or more non-hexose sugar alcohols, such as xylitol, xylose, erythritol, ribose, and/or arabinose, may be used. For example, in some embodiments and implementations, the composition may comprise at least about 0.5% by weight of xylitol, xylose, erythritol, erythrose, ribose, ketose, and/or arabinose. In some such embodiments and implementations, the composition may comprise at least about 2% by weight of xylitol, xylose, erythritol, erythrose, ribose, ketose, and/or arabinose. In some such embodiments and implementations, the composition may comprise between about 0.5% and about 50% by weight of xylitol, xylose, erythritol, erythrose, ribose, ketose, and/or arabinose. In some such embodiments and implementations, the composition may comprise between about 1% and about 30% by weight of xylitol, xylose, erythritol, erythrose, ribose, ketose, and/or arabinose. In some such embodiments and implementations, the composition may comprise between about 5% and about 20% by weight of xylitol, xylose, erythritol, erythrose, ribose, ketose, and/or arabinose.

In certain preferred embodiments and implementations, any of the above-referenced combinations/concentrations of xylitol, xylose, erythritol, erythrose, ribose, ketose, and/or arabinose may be limited to xylitol, xylose, and/or erythritol, which may have some of the most beneficial properties described herein. Similarly, in certain other embodiments and implementations, any of the above-referenced combinations/concentrations of xylitol, xylose, erythritol, erythrose, ribose, ketose, and/or arabinose may be limited to xylitol and/or erythritol. In some such embodiments and implementations, any of the above-referenced combinations/concentrations of xylitol, xylose, erythritol, erythrose, ribose, ketose, and/or arabinose may be more specifically limited to use of xylitol alone (along with one or more antihistamines and other suitable inactive ingredients).

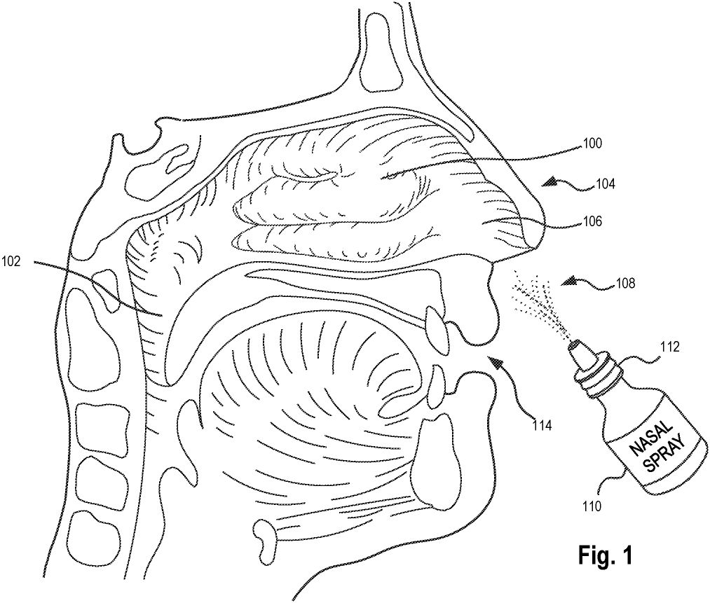

The compositions disclosed herein may be delivered via an intranasal pathway. For example, in some implementations, the composition may be delivered to an individual’s nasopharnyx via a nasal spray. FIG. 1 illustrates intranasal delivery of a composition 108 comprising, for example, xylitol and one or more antihistamines consistent with embodiments disclosed herein.

Anatomically, the nasopharynx 100 is a point at which the nasal passages 106 merge into one. It is also where the floor of the nose 104 bends downward with the superior-posterior surface of the palate. The openings of the auditory tubes (i.e., eustachian tubes) and the posterior nasal apertures (i.e., choanae) are located within the nasopharynx 100. The oropharynx 102 is located inferior to the nasopharynx 100 and is behind the mouth 114. By virtue of the anatomic locations of the eustachian tube openings in the nasopharynx 100, nasal administration of a solution or other composition may result in a more effective exposure of the eustachian tube openings versus administration via an oral route. Accordingly, administering the composition 108 via an intranasal pathway may be more effective than other routes of administration (e.g., orally, topically). However, it is anticipated that in other embodiments and implementations, one or more of the compositions disclosed herein may alternatively be administered orally by way of drops, a spray, a gel, a solution, or the like.

In certain embodiments, administering the composition 108 via an intranasal pathway may be performed utilizing a nasal spray bottle 110. The nasal spray bottle 110 may any suitable bottle configured to retain compositions 108 including one or more antihistamines, such as brompheniramine, cetirizine, clemastine, diphenhydramine, fexofenadine, and/or loratadine, along with xylitol or another of the non-hexose sugar alcohols disclosed herein, and to distribute (e.g., spray) the composition 108 into an individual’s intranasal pathway and/or nasopharynx 100 using a pump mechanism 112. Alternatively, the composition may be delivered from other nasal spray bottles by simply squeezing the bottle. In certain embodiments, the composition 108 may be stored in the nasal spray bottle 110 in liquid or powder form, and may be distributed into the intranasal pathway and/or nasopharnyx 100 as an aerosol.

In some embodiments, the composition 108 may be administered using a bathing delivery method. A bathing delivery method may utilize a solution comprising the composition 108 including one or more of brompheniramine, cetirizine, clemastine, diphenhydramine, fexofenadine, and loratadine, preferably along with xylitol, contained within a dilute (e.g., a less viscous, more fluid composition), pharmaceutically acceptable carrier suitable for nasal administration.

For example, the composition 108 may be contained within an aqueous solution including water and/or other pharmaceutically-acceptable carrier components. In certain embodiments, the composition may comprise approximately 0.1% saturation of a suitable aqueous solution. In certain embodiments, the composition may comprise approximately 1-15% of a suitable aqueous solution.

The bathing delivery method may directly deliver the composition 108 to the nasopharynx 100 in conjunction with subsequent bathing of the nasopharyngal area. As discussed above, this may be achieved using a nasal spray bottle 110. However, in alternative implementations, the composition may be delivered using alternative delivery mechanisms, such as droppers, misters, atomizers, brushes, swabs, etc. In some embodiments, utilizing a free-flowing, low viscosity aqueous solution may allow for a rapid and concentrated application of the composition.

In further embodiments, the composition 108 may be administered using an adhesion delivery method. An adhesion delivery method may utilize a more viscous solution, such as a gel, to deliver the composition 108. In certain embodiments, the viscous solution may include a bio-adhesive agent. An adhesion delivery method may rely on the adhesion of a solution containing the composition 108 to the nasal mucous membrane and a slow migration of the solution to the nasopharyngeal area. Utilizing a more viscous solution may provide for a more gradual and steady application of the composition to desired areas, such as within the nasal cavity, and may also increase the duration of the positive benefits of the composition, such as by decreasing the rate at which the composition is removed from the desired areas.

In certain embodiments, a solution including the composition 108 may include a buffer, a thickening agent, a bio-adhesive, and/or a humectant. A pharmaceutically acceptable surfactant and a preservative may also be included along with one or more excipients suitable for a pharmaceutical composition.

In embodiments including a buffer, the buffer may be configured to maintain a pH level of the solution. Exemplary suitable buffers include acetate, citrate, and phosphate buffers. The thickening agent may include, for example, one or more of methylcellulose, xanthan gum, carboxyl methylcellulose, polyvinyl alcohol, hydroxpropyl cellulose, carbomer, starches, chitosans, acrylates, and mixtures thereof. In certain embodiments, these substances may also act as suitable bio-adhesives. Suitable exemplary humectants include sorbitol, propylene glycol, glycerol, and/or any combination thereof. Suitable surfactants may be anionic, cationic, or nonionic, and may include polyoxyethylene derivatives, fatty acids, and/or partial esters of sorbitol anhydrides. For example, the surfactant may include sodium lauryl sulfate, polysorbate 80, polyoxyl Stearate, polyoxy ethylene 50, fusieates, bile salts, and octoxynol. However, it should also be understood that many embodiments of the compositions disclosed herein will not need to include a buffer.

In preferred implementations of methods for treating or preventing nasal, ear, and/or sinus infections, the composition may be administered into the nasal passage of a subject, such as by way of a nasal spray or other suitable applicator, at a frequency rate of one or two sprays in each nostril from once daily to four times daily. In a most preferred implementation, the treatment regimen may comprise administration of the composition into the nasal passage of a subject at a frequency rate of one or two sprays in each nostril three times daily, in some such implementations until the bottle is empty.

The preferred dosage range used in connection with one or more the embodiments and/or implementations disclosed herein may be from about 0.1 mL to about 0.6 mL of fluid per dose.

It will be understood by those having skill in the art that changes may be made to the details of the above-described embodiments without departing from the underlying principles presented herein. For example, the anti-microbial and antihistamine compositions disclosed herein may be administered via liquid drops from a dropper, topically (in some cases using a cotton swab or the like), orally, via a mister or atomizer, and/or via any other suitable manner of administration. In addition, any suitable combination of various embodiments, or the ingredients, methods, or features thereof, is contemplated.

Any methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified. It should also be understood that some implementations can be practiced without some or all of the steps disclosed herein. In addition, the steps of a method do not necessarily need to be executed in any specific order, or even sequentially, nor need the steps be executed only once, unless otherwise specified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment.

It will be apparent to those having skill in the art that changes may be made to the details of the above-described embodiments without departing from the underlying principles set forth herein. The scope of the present inventions should, therefore, be determined only by the following claims.

1. A method for treatment of a bacterial infection using a nasal composition, wherein the method comprises the steps of:
identifying a subject having a bacterial infection; and
delivering a dose of a composition into the subject’s nasal passage, wherein the composition comprises:
an antihistamine comprising at least one of brompheniramine,
cetirizine, clemastine, diphenhydramine, fexofenadine, and loratadine; and
at least one of xylitol and xylose.

2. The method of claim 1, wherein the antihistamine is present in the composition in a therapeutically effective concentration for treating an allergy condition in the subject.

3. The method of claim 2, wherein the antihistamine is present in the composition in a concentration of between about 0.01% and about 2% by weight.

4. The method of claim 3, wherein the antihistamine is present in the composition in a concentration of between about 0.2% and about 2% by weight.

5. The method of claim 1, wherein the at least one of xylitol and xylose is present in the composition in a therapeutically effective concentration for treating the bacterial infection.

6. The method of claim 5, wherein the at least one of xylitol and xylose is present in the composition in a concentration of between about 5% and about 20% by weight.

7. The method of claim 1, wherein the bacterial infection comprises a bacterial biofilm.

8. The method of claim 7, wherein the composition disrupts the bacterial biofilm.

9. The method of claim 1, wherein the dose is part of a treatment regimen, and wherein the treatment regimen comprises delivering the composition in each nostril of the subject three times daily for at least ten days.

10. A method for treatment of a bacterial infection using a nasal composition, wherein the method comprises the steps of:
identifying a subject having a bacterial infection, wherein the bacterial infection comprises a bacterial biofilm; and
delivering a dose of a composition into the subject’s nasal passage, wherein the composition comprises:
an antihistamine comprising at least one of brompheniramine, cetirizine, clemastine, diphenhydramine, fexofenadine, and loratadine; and
at least one non-hexose sugar alcohol in a in a therapeutically effective concentration for treating the bacterial infection.

11. The method of claim 10, wherein the at least one non-hexose sugar alcohol comprises at least one of xylitol, xylose, erythritol, erythrose, ribose, ketose, and arabinose.

12. The method of claim 11, wherein the at least one non-hexose sugar alcohol is present in the composition in a concentration of between about 5% and about 20% by weight.

13. The method of claim 11, wherein the at least one non-hexose sugar alcohol comprises xylitol.

14. The method of claim 13, wherein the xylitol is present in the composition in a concentration of at least 2% by weight.

15. The method of claim 14, wherein the xylitol is present in the composition in a concentration of between about 5% and about 20% by weight.

16. A method for disruption of a bacterial biofilm using a nasal composition, wherein the method comprises the steps of:
identifying a subject having a bacterial infection comprising a bacterial biofilm; and
delivering a dose of a composition into the subject’s nasal passage,
wherein the composition comprises xylitol in a in a concentration of at least 2% by weight.

17. The method of claim 16, wherein the composition further comprises an antihistamine.

18. The method of claim 17, wherein the antihistamine comprises at least one of brompheniramine, cetirizine, clemastine, diphenhydramine, fexofenadine, and loratadine.

19. The method of claim 16, wherein the xylitol is present in the composition in a concentration of between about 5% and about 20% by weight.

20. The method of claim 16, wherein the composition further comprises at least one of xylose, erythritol, erythrose, ribose, ketose, and arabinose.
